(19) United States
(12) Patent Application Publication
(10) Pub. No.: US 2020/0179553 A1
(43) Pub. Date: Jun. 11, 2020

(54) SHOWER DIFFUSER

(71) Applicant: Austin Ferguson Young, Lake Placid, NY (US)

(72) Inventor: Austin Ferguson Young, Lake Placid, NY (US)

(21) Appl. No.: 16/210,450

(22) Filed: Dec. 5, 2018

(51) Int. Cl. A61L 9/12; A47K 3/28

(52) U.S. Cl. CPC A61L 9/122; A47K 3/281; A61L 9/127

(57) ABSTRACT

A portable shower diffuser. The portable shower diffuser includes a housing. A vent, such as a plurality of vent openings, are defined through a portion of the housing. The housing further includes a wick receptacle configured to retain a wick within the housing. A power source is coupled to the housing. An electric blower is disposed within the housing. A switch electrically connects the power source to the electric blower. The electric blower includes a blower vent positioned to blow air through the wick receptacle and out of the vent.

### BACKGROUND OF THE INVENTION

The present invention relates to diffusers of essential oils and, more particularly, to a shower diffuser that is portable.

Aromatherapy uses plant materials and aromatic plant oils, including essential oils, and other aroma compounds for improving psychological or physical well-being. Diffusers are used to diffuse essential oils in aromatherapy. Currently, there are no affordable and practical diffusers made to use in the shower.

As can be seen, there is a need for an affordable and portable shower diffuser.

### SUMMARY OF THE INVENTION

In one aspect of the present invention, a portable shower diffuser comprises: a housing comprising a vent defined through a portion of the housing and a wick receptacle configured to retain a wick within the housing; a power source coupled to the housing; an electric blower disposed within the housing; and a switch electrically connecting the power source to the electric blower, wherein the electric blower is positioned to blow air through the wick receptacle and out of the vent.

These and other features, aspects and advantages of the present invention will become better understood with reference to the following drawings, description and claims.

### BRIEF DESCRIPTION OF THE DRAWINGS

FIG. 1 is a perspective view of an embodiment of the present invention shown in use;

FIG. 2 is a front view of an embodiment of the present invention, with a front wall removed to illustrate internal components;

FIG. 3 is a side view taken along line 3-3 in FIG. 2;

FIG. 4 is a bottom view taken along line 4-4 in FIG. 2;

FIG. 5 is an exploded view of an oil diffuser wick of an embodiment of the present invention; and

FIG. 6 is a side view of a resealable cap of the oil diffuser wick of an embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

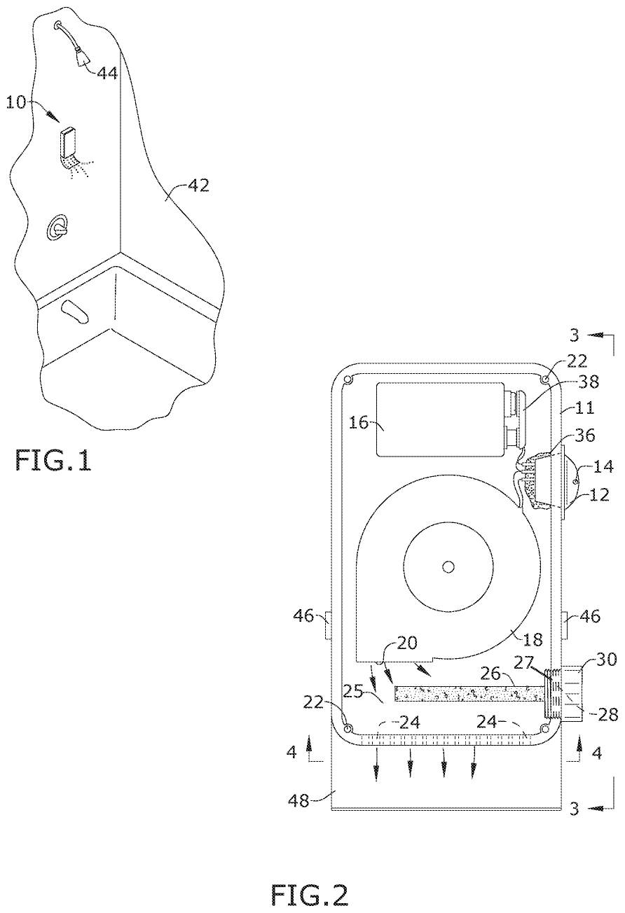

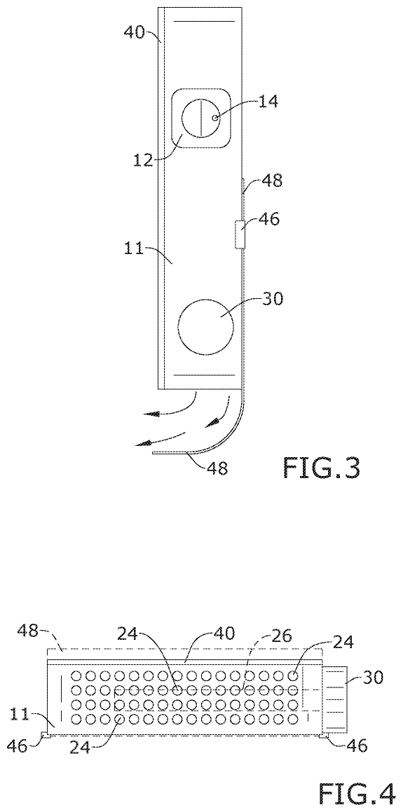

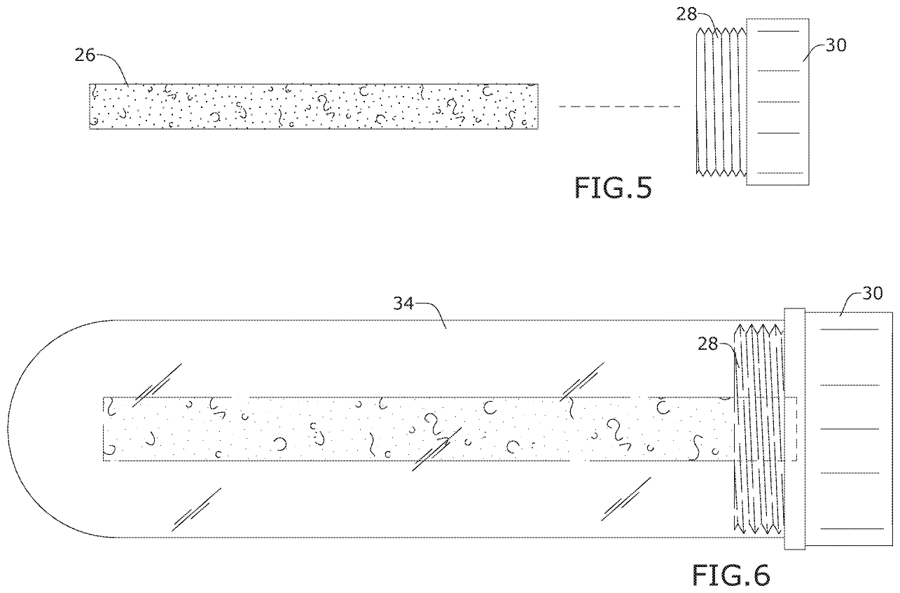

Referring to FIGS. 1 through 6, the present invention includes a portable shower diffuser 10. The portable shower diffuser 10 includes a housing 11. A vent 24, such as a plurality of vent openings, are defined through a portion of the housing 11. The housing 11 further includes a wick receptacle 25 configured to retain a wick 26 within the housing 11. A power source 16, 38 is coupled to the housing 11. An electric blower 18 is disposed within the housing 11. A switch 12 electrically connects the power source 16, 38 to the electric blower 18. The electric blower 18 includes a blower vent 20 positioned to blow air through the wick receptacle 25 and out of the vent 24.

The housing 11 includes a front wall 40, a rear wall, a top wall, a bottom wall, and sidewalls. The front wall 40 may be detachable using screws and screw openings 22 to access the internal components of the diffuser 10. The housing 11 may be made of a waterproof plastic material. A hook and loop adhering strip, suctions cups, or other connectors may connect the rear wall of the housing 11 to a shower wall 42. For example, the housing 11 may be coupled below the shower head 44. The vent 24 is defined through the bottom wall. The rear wall may further include clips 46.

In certain embodiments, the present invention includes a curved deflector 48. The curved deflector 48 may be coupled to the rear and extend downward from the rear wall. The curved deflector 48 curves towards the bottom wall and is disposed below the vent 24. The curved deflector 48 deflects air from the vent towards a user.

The switch 12 may include a toggle switch extending outside of the housing 11. The switch 12 may further include a light 14 indicating when the switch is toggled to the on position. In certain embodiments, the power source 16, 38 includes battery connectors 38 disposed within the housing 11. A battery 16, such as a 9-volt battery, is coupled to the battery connectors 38 to power the electric blower 18 when the switch 12 is toggled on. The electric blower 18 may include a brushless blower fan. Hot glue 36 may be used to seal and waterproof the circuitry.

In certain embodiments, the housing 11 includes a coupler 27 configured to retain the wick 26 within the wick receptacle 25. The coupler 27 may include an opening having a female threading. However, the coupler 27 may include any type of coupler that secures the wick 26 to the housing 11, such as but not limited to, clips, buckles, clamps, and the like.

The wick 26 of the present invention is an absorbent material, such as cotton, to retain essential oils and then disperse the essential oils when the blower 20 blows air on the wick 26. The present invention may further include a wick handle 30. The wick 26 extends from the wick handle 30. The wick handle 30 may include a male threading 28 that mechanically fastens to the female threading, thereby retaining the wick 26 within the wick receptacle 25. When the wick 26 is not in use, the wick 26 may be stored within a torpedo shaped cap 34 to prevent the wick 26 from drying out.

A method of making the present invention may include the following. Acquire plastic waterproof housing for the internal components. Drill a hole into the side of the box shaped unit for the toggle switch. Affix the blower fan inside the box about central portion. Wire and solder the blower-fan and battery to the toggle switch in the appropriate positions and adhere the blower fan with hot glue. Waterproof the exposed toggle posts and soldered wire with a generous amount of hot glue so that none of the conductive metal is exposed. Drill another hole under the toggle switch on the side for the torpedo shaped aroma therapy insert. Finally, on the bottom of the unit drill multiple small vent holes for the blower fan to excrete the aroma through.

It should be understood, of course, that the foregoing relates to exemplary embodiments of the invention and that modifications may be made without departing from the spirit and scope of the invention as set forth in the following claims.

What is claimed is:

1. A portable shower diffuser comprising:
a housing comprising a vent defined through a portion of the housing and a wick receptacle configured to retain a wick within the housing;
a power source coupled to the housing;
an electric blower disposed within the housing; and
a switch electrically connecting the power source to the electric blower, wherein
the electric blower is positioned to blow air through the wick receptacle and out of the vent.

2. The portable shower diffuser of claim 1, wherein the housing comprises a front wall, a rear wall, a top wall, a bottom wall, and sidewalls.

3. The portable shower diffuser of claim 2, wherein the vent is defined through the bottom wall.

4. The portable shower diffuser of claim 3, further comprising a curved deflector extending downward from the rear wall and curving towards the bottom wall.

5. The portable shower diffuser of claim 1, wherein the switch comprises a toggle switch extending outside of the housing.

6. The portable shower diffuser of claim 1, wherein the power source comprises battery connectors disposed within the housing.

7. The portable shower diffuser of claim 1, further comprising a coupler configured to retain the wick within the wick receptacle.

8. The portable shower diffuser of claim 7, wherein the coupler is an opening comprising a female threading.

9. The portable shower diffuser of claim 8, further comprising the wick, wherein the wick is an absorbent material.

10. The portable shower diffuser of claim 9, further comprising a wick handle, wherein the wick extends from the wick handle, wherein the wick handle comprises a male threading mechanically fastened to the female threading.
